(19) United States
(12) Patent Application Publication
(10) Pub. No.: US 2011/0098579 A1
(43) Pub. Date: Apr. 28, 2011

(54) BLOOD-PRESSURE SENSOR SYSTEM

(75) Inventors: Yoshiharu AJIKI, Hachioji-shi (JP); Isao SHIMOYAMA, Tokyo (JP); Kiyoshi MATSUMOTO, Tokyo (JP)

(73) Assignees: OLYMPUS CORPORATION, Tokyo (JP); THE UNIVERSITY OF TOKYO, Tokyo (JP)

(21) Appl. No.: 12/894,921

(22) Filed: Sep. 30, 2010

(30) Foreign Application Priority Data
Oct. 26, 2009 (JP) 2009-245741
Jul. 6, 2010 (JP) 2010-154322

(51) Int. Cl. A61B 5/021

(52) U.S. Cl. 600/485

(57) ABSTRACT

There is provided a blood-pressure sensor system wherein a blood-pressure sensor including a plurality of structures which induces surface plasmon resonance on a light-receiving plane of a photoelectric conversion element is attached to an outer wall of a blood vessel, in which if the blood-pressure sensor is deformed according to expansion or contraction of the blood vessel, an interval of layout of the structure is changed (widened or narrowed), so that an incident form of light with respect to the structures is changed to cause the output from the photoelectric conversion element to be changed. The output is measured as an open circuit voltage, and index calculation is performed, so that a blood pressure value is measured.

### CROSS-REFERENCE TO RELATED APPLICATIONS

This application is based upon and claims the benefit of priority from prior. Japanese Patent Applications No. 2009-245741, filed Oct. 26, 2009; and No. 2010-154322, filed Jul. 6, 2010, the entire contents of both of which are incorporated herein by reference.

### BACKGROUND OF THE INVENTION

1. Field of the Invention

The present invention relates to a blood-pressure sensor system of optically measuring mechanical attributes such as a displacement of a measurement object and a force and measuring an internal pressure of a blood vessel from the displacement of the blood vessel.

2. Description of the Related Art

As a blood-pressure sensor that is generally known as a tonometer, there are two types, that is, a direct type and an indirect type. In the direct type, a sensor is inserted into an internal part of a blood vessel of a test object to measure a blood pressure value. The indirect type is classified into an invasive type or a non-invasive type, where a sensor is disposed near the blood vessel to indirectly measure the blood pressure value. Among these types, the direct type is a measurement method used upon surgery or the like, and the non-invasive type is a simple measurement method used with ease in general households.

As a sensor for use in direct measurements, there is known an implant type blood-pressure sensor which can always measure the blood pressure so as to allow monitoring of the blood pressure of a patient suffering from, for example, a vascular disease such as a congestive heart disease or an arteriolosclerosis or monitoring of the blood pressure of a patient after artificial blood vessel replacement.

### BRIEF SUMMARY OF THE INVENTION

According to an embodiment of the present invention, there is provided a blood-pressure sensor system comprising a blood-pressure sensor including:

a photoelectric conversion element which converts light incident to a light-receiving plane into an electrical signal and outputs the electrical signal; and a plurality of structures which are separately disposed on the light-receiving plane at predetermined intervals and each of which is made of a conductive material that induces surface plasmons, wherein when the blood-pressure sensor is attached to an outer wall of a blood vessel, the intervals of the structures are changed to expanding an opening or to be tapered according to expansion or contraction of the blood vessel due to a change in the blood pressure of the blood vessel, so that a form of light incident to the structures is changed to cause a change in the output from the photoelectric conversion element, and index calculation is performed on the output measured as an open circuit voltage, and a blood pressure value is obtained using a result of the index calculation and information where the indexes and the blood pressure values are associated with each other.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram illustrating a configuration of a blood-pressure sensor system according to a first embodiment of the invention;

FIGS. 2A, 2B, 2C, 2D, and 2E are diagrams illustrating an example of a configuration of a blood-pressure sensor in the blood-pressure sensor system;

FIGS. 3A, 3B, 3C, 3D, 3E, 3F, and 3G are process diagrams for explaining processes of manufacturing the blood-pressure sensor;

FIG. 4 is a characteristic diagram illustrating a relationship between wavelength and light absorbance in a plurality of structures which induce surface plasmons;

FIG. 5 is a diagram illustrating a relationship between a rate β and a pitch a;

FIG. 6 is a diagram illustrating a change in pressure (AOP) [mmHg] of a blood vessel and a change of an amount of strain (1−β) with time;

FIG. 7 is a diagram illustrating a relationship between a pitch and a rate at the time of measuring a pressure by using the blood-pressure sensor according to the embodiment at another external temperature;

FIG. 8 is a block diagram illustrating a configuration of a blood-pressure sensor system according to a second embodiment of the invention;

FIG. 9A is a diagram illustrating a layout configuration of a blood-pressure sensor 2A illustrated in FIG. 8, and FIG. 9B is a cross-sectional view illustrating a configuration of the blood-pressure sensor 2A taken along line IXB-IXB of FIG. 8;

FIG. 10 is a block diagram illustrating a configuration of a blood-pressure sensor system according to a third embodiment of the invention;

FIG. 11 is a diagram illustrating a layout configuration of a blood-pressure sensor 2B illustrated in FIG. 10;

FIG. 12 is a cross-sectional view illustrating a configuration of a blood-pressure sensor system according to a fourth embodiment of the invention;

FIG. 13 is a diagram illustrating a configuration of a system of investigating an angle dependency of incident light in an elastic grating structure;

FIGS. 14A and 14B are diagrams illustrating a configuration and a slope of an elastic grating structure, FIG. 14C is a diagram illustrating an output based on primary reflecting light in a sample table that is slanted, and FIG. 14D is a diagram illustrating an estimated value θ_{R} of an angle of rotation and an estimated value obtained by using a grating equation;

FIG. 15 is a diagram illustrating outputs of reflecting light at an incident angle in a first sample and a second sample;

FIG. 16A is a diagram illustrating an example of a laminate structure of the first sample, and FIG. 16B is a diagram illustrating an example of a laminate structure of the second sample;

FIG. 17 is a diagram illustrating a relationship between a stage rotation angle and an open circuit voltage at an estimated value (angle) of occurrence of surface plasmon resonance;

FIG. 18 is a diagram illustrating a relationship between an incident angle and an open circuit voltage in a solar cell structure comprising an elastic grating structure;

FIG. 19 shows the relationships between a incident angle and the zero-order reflecting light, and between incident angle and open circuit voltage and

FIG. 20 is a diagram illustrating a relationship between an open circuit voltage and a pitch L according to a modified example of the fourth embodiment.

### DETAILED DESCRIPTION OF THE INVENTION

Hereinafter, exemplary embodiments of the invention will be described with reference to the accompanying drawings.

First, concept of a blood-pressure sensor system according to the embodiments of the invention is described.

A blood-pressure sensor according to the embodiment is a mechanical sensor capable of grasping mechanical attributes of an object. The blood-pressure sensor measures an internal pressure of a blood vessel (hereinafter, referred to as a blood pressure or a blood pressure value) outside a blood vessel.

The blood-pressure sensor is configured to have a structure which induces surface plasmon resonance (SPR) on a light-receiving plane of a photoelectric conversion element such as a solar cell. Electrical data (charge amount) output from the photoelectric conversion element is compared with the data which are set in correspondence with blood pressure values in advance, to be converted into a corresponding blood pressure value, and the blood pressure value is output. In addition, if the blood-pressure sensor is deformed by an externally applied force, a change (expansion or contraction) in the interval of the plurality of the structures, that is, a change in a layout occurs. In other words, an incident form of light with respect to the structures changes, which causes a change in the electrical data (charge amount) output from the photoelectric conversion element. The change is converted into the blood pressure value, and information of the change in the blood pressure is acquired.

The detection in the sensor unit is performed by measuring an open circuit voltage of the solar cell and performing an index calculation, so that a displacement (blood pressure value) of the object is measured from the information. In other words, in order to measure the open circuit voltage, no current is flown into the sensor unit (although an infinitesimal current for voltage measurement is flown), and heat is prevented from being generated.

In general, a photoelectric conversion element is used as a power source element or a solid state image pickup element by generating electric energy (electric charges) from received light like a power generating element or an image pickup element. However, in the embodiment, the photoelectric conversion element is not limited to the power generating element or the solid state image pickup element. The photoelectric conversion element has a structure which induces the surface plasmon resonance. If an external force is applied, the photoelectric conversion element is deformed, so that an incident form of light with respect to the structure is changed. As a result, a change in the generated charge amount occurs. The photoelectric conversion element can be used as a mechanical sensor using the change. The surface plasmon effect is well known, and there are disclosed several structures for obtaining the effect. The structure of the blood-pressure sensor according to the embodiment is also designed by using parameters in the case of using a blood vessel as a test object.

In the below description, terms and symbols (parameters) are denoted as follows.

(a) Mechanical Characteristic: A characteristic representing strain, force, pressure, or the like.

Particularly, in the embodiment, as an example, a blood pressure value is a measurement object.

(b) Grating Structure: Denotes a structure where structures 1 inducing surface plasmon are arrayed in a shape of a grating,

(c) Voc: Open Circuit Voltage, which is generated when a load is not connected to a solar cell,

(d) Isc: Short Circuit Current, which flows when the solar cell is short-circuited,

(e) Io: Leakage Current, which flows when the solar cell is in a dark state,

(f) L: Pitch between the structures 3,

(g) a: Half value of L (=L/2),

(h) b: Width of the structure 3,

(i) c: Height of the structure 3,

(j) k: Boltzmann Constant (1.38066×10⁻²³[J/K]),

(m) T: Temperature [K],

(n) q: Elementary Charge (1.60218×10⁻¹⁹[C]),

(o) α: Ratio of Isc to Io (Isc/Io),

(r) β: Ratio of α to a minimum α0 of the ratio α,

(s) Pmin: Minimum Value of Blood Pressure,

(t) Paorta: Internal Pressure of Blood Vessel,

(u) η: Pressure Conversion Coefficient (Coefficient constructed with Young's modulus of blood vessel and Poisson's Ratio),

(x) w, d, and h: Width, length, and height of a sensor,

(Y) W and D: Width and length of a sensor and a peripheral circuit unit.

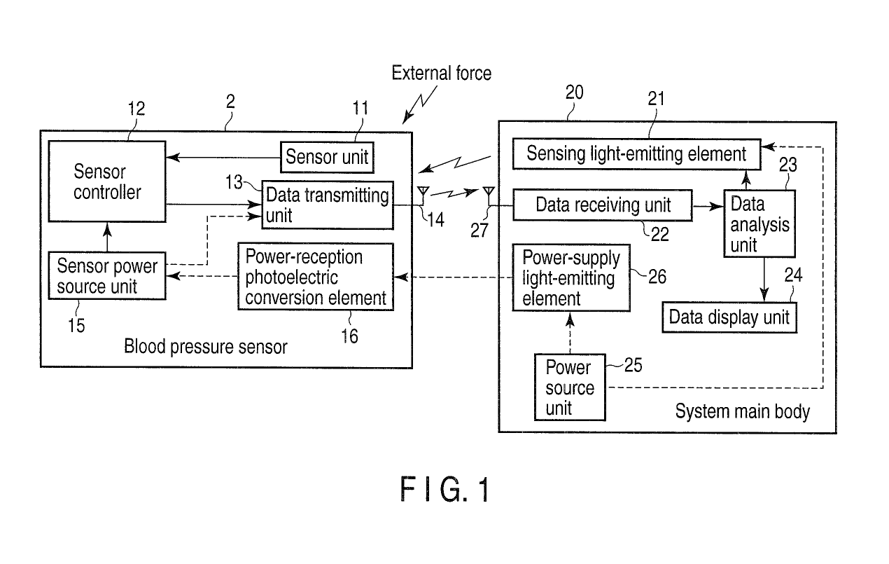

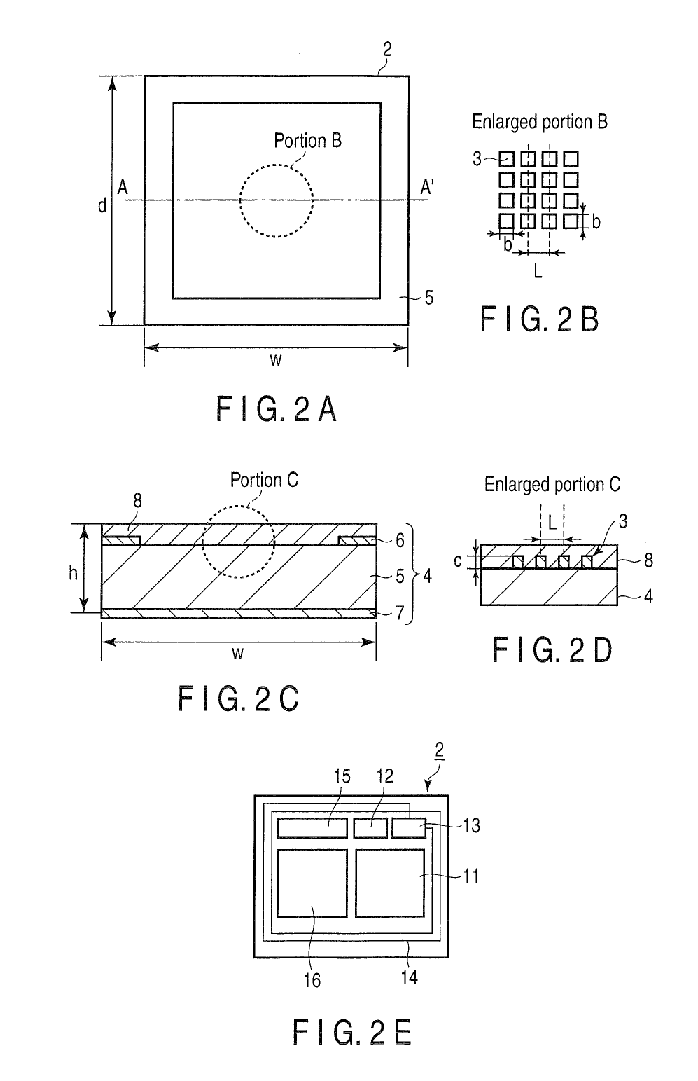

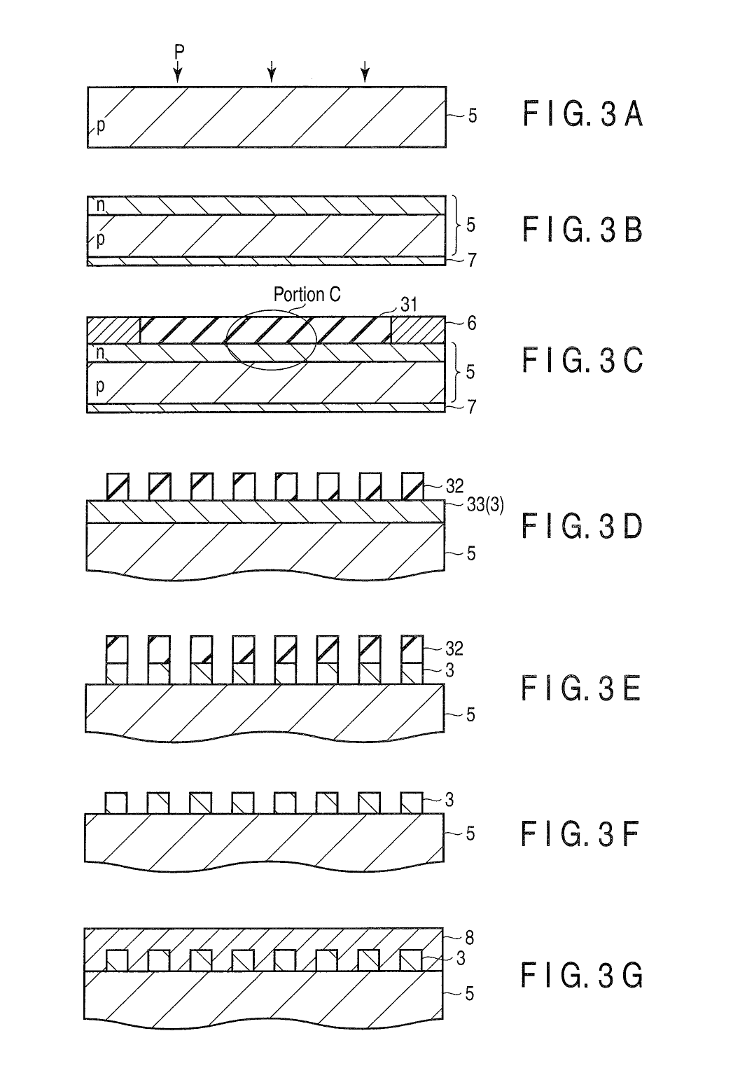

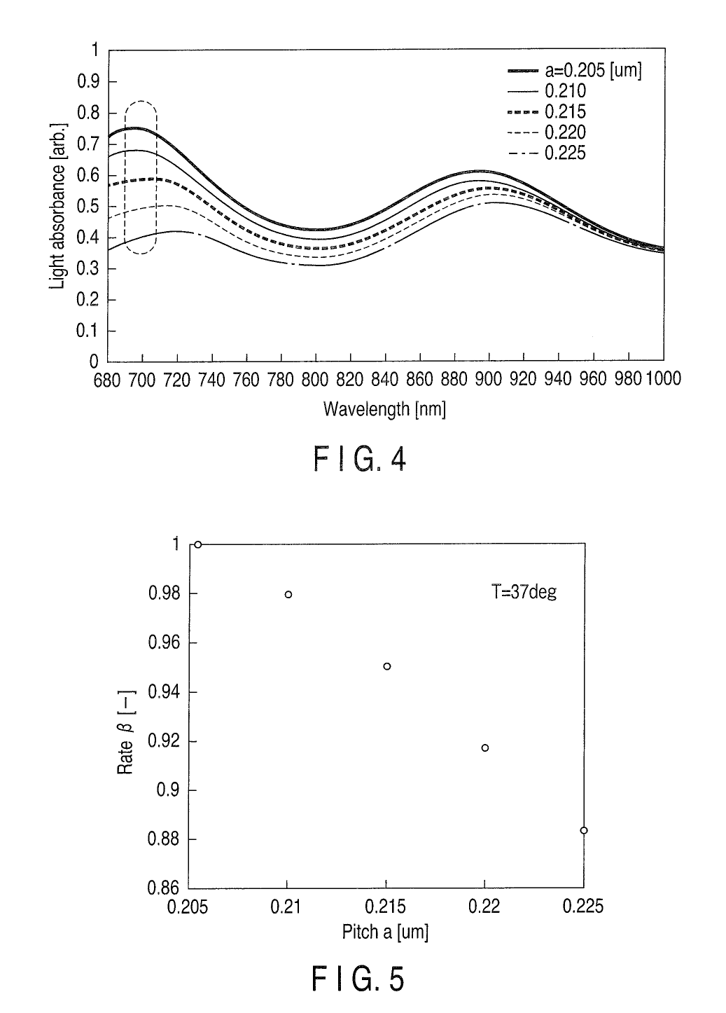

FIG. 1 is a block diagram illustrating a configuration of a blood-pressure sensor system according to a first embodiment of the invention. FIGS. 2A, 2B, 2C, 2D, and 2E are diagrams illustrating an example of a configuration of a blood-pressure sensor in the blood-pressure sensor system. FIGS. 3A, 3B, 3C, 3D, 3E, 3F, and 3G are process diagrams for explaining processes of manufacturing the blood-pressure sensor. FIG. 4 is a characteristic diagram illustrating a relationship between a wavelength and a light absorbance in a plurality of structures which induce surface plasmons.

FIG. 1 illustrates an example of a configuration of the blood-pressure sensor system according to the embodiment.

The blood-pressure sensor system 1 includes a blood-pressure sensor 2 and a system main body 20 which are separate components. The blood-pressure sensor 2 is attached to an outer wall of a blood vessel. Data transmission between the blood-pressure sensor 2 and the system main body 20 is performed by using electromagnetic induction communication or radio communication. Power supply to the blood-pressure sensor is performed by using light.

The blood-pressure sensor 2 comprises a sensor unit 11, a sensor controller 12, a data transmitting unit 13, a data-transmission antenna 14, a sensor power source unit 15, and a power-reception photoelectric conversion element 16.

The sensor unit 11 mainly comprises a later-described photoelectric conversion element 2 and structures 3 which induce surface plasmons. If the sensor unit 11 receives light having an arbitrary wavelength A, the output of the charge amount relatively linearly changes due to the photoelectric conversion. When the sensor is deformed by an externally applied force, the charge amount is changed.

The sensor controller 12 acquires a voltage value (open circuit voltage value) based on the charge amount generated by the sensor unit 11, converts the voltage value into a data signal for communication, and transmits the data signal to the data transmitting unit 13.

The data transmitting unit 13 converts the estimated blood pressure value into a communication signal suitable for electromagnetic induction communication or radio communication and transmits the communication signal through the data-transmission antenna 14. In the embodiment, as a communication frequency in the electromagnetic induction communication, by considering a permeability of a living body and a size thereof, a frequency near 433 [MHz] is used as an example. The sensor power source unit 15 converts the power source generated by the power-reception photoelectric conversion element 16 into a driving power source and supplies the driving power source to the data transmitting unit 13 and the sensor controller 12.

In addition, the system main body 20 comprises s a sensing light-emitting element 21 including a light-emitting diode or the like which emits light-for-measurement to the blood-pressure sensor 2, a data receiving unit 22 which receives data transmitted from the data transmitting unit 13 through an antenna 27 and decodes the data, a data analysis unit 23 which analyses received data and performs image signal processing to reproduce a blood pressure value (numerical data), a data display unit 24 including a monitor which displays the obtained blood pressure value, information thereon, manipulation, instructions, and the like, a power source unit 25 which is constructed with a battery or the like, and a power-supply light-emitting element 26 including a light-emitting diode or the like which converts power supplied from the power source unit 25 to light through photoelectric conversion.

The light emitted from the sensing light-emitting element 21 may be configured to be superposed with the light for transmission of a control signal (operation instruction) as well as the light-for-measurement for measurement of the blood-pressure sensor 2 in a communicable manner (or in a manner of communication provided with a separate communication period).

The data analysis unit 23 estimates a deformation amount through a later-described calculation process and obtains an estimated blood pressure value from a relationship between the deformation amount and blood pressure values which are acquired empirically (through experiment, simulation, or the like) in advance and listed in a table. In addition, a calculation program and a data table may be stored in a rewritable memory (not shown), for example, a flash memory and be rewritten by performing updating or the like if needed. In addition, the data analysis unit 23 also includes a controller which controls the entire system. In addition, although not shown, an I/O interface mechanism which communicates with an external apparatus to change or exchange the analyzed data or condition settings (parameters or programs) may be provided.

In such a configuration, a light-emitting diode (bomb type LED) of, for example, infrared light of 720 [nm] is used as the sensing light-emitting element 21 for performing the data communication, and a light-emitting diode (bomb type LED) of, for example, 810 [nm] is used as the power-supply light-emitting element 26. In addition, as the timing of performing the communication, the sensor unit 11 may always perform detecting the blood pressure and the communication may be performed in a predetermined period. In addition, when the blood pressure value is changed, the communication may be performed.

Next, a configuration of the blood-pressure sensor 2 and processes of manufacturing the blood-pressure sensor 2 are described.

FIG. 2A is a diagram illustrating an outer configuration of the blood-pressure sensor according to the embodiment as viewed from the top side. FIG. 2B is an enlarged diagram illustrating the structures, which induce the surface plasmons, formed in the grating structure. FIG. 2C is a diagram illustrating a configuration of a cross section of the blood-pressure sensor taken along line A-A′ of FIG. 1. FIG. 2D is an enlarged diagram illustrating a portion C illustrated in FIG. 2C as a configuration of a cross section of the grating structure where the structures which induce the surface plasmons are disposed. FIG. 2E is a diagram illustrating a configuration of layout of a blood-pressure sensor system.

As illustrated in FIGS. 2A and 2C, the blood-pressure sensor 2 includes a photoelectric conversion element 4, a plurality of structures 3 which are disposed on the surface of the photoelectric conversion element 4 and constructed with a conductive material to induce surface plasmons, and a transparent (or semi-transparent) protective layer 8 which is the uppermost layer to cover at least the structures 3. The protective layer 8 is constructed with, for example, a transparent conductive material. Each circuit element is formed on one-chip substrate. In the embodiment, the size of the blood-pressure sensor 2 is assumed to be about 2 mm square.

The photoelectric conversion element 2 includes a substrate 5 which is constructed with a pn type semiconductor, a charge collecting layer 6 which is disposed to surround the structures 3 disposed on a front surface of the substrate 5 in a rectangular shape, and a rear surface charge collecting layer 7 which is disposed on a rear surface of the substrate 5. The photoelectric conversion element 2 is a well-known component and is constructed with a pn junction of semiconductor and electrodes. As a semiconductor material for the substrate 5, a material having the highest efficiency according to a wavelength range of receiving light is selected.

Now, the structure 3 which induces the surface plasmons is described.

As illustrated in FIGS. 2B and 2D, rectangular structures 3 are formed to be surrounded by the charge collecting layer 6 on the photoelectric conversion element 2 in the grating structure. Each of the rectangular structures 3 is formed in a square shape having the same horizontal and vertical widths b and a height c. The structures 3 are disposed in a matrix shape with a distance L between the centers thereof.

In the structure 3 according to the embodiment, with respect to the value a (=L/2), a blood vessel deforming strain which is (maximum blood pressure value)−(minimum blood pressure value) is set to 10% by using a rigorous coupled wave analysis (RCWA). In the embodiment, as dimensions of the structure 3 having a cubic structure, both of the width b and the height c are set to 150 [nm] in the calculation. As a result, it is possible to obtain a=0.205 to 0.225 [μm] as illustrated in FIG. 4. Therefore, as an initial pattern before the deformation, a=0.205 μm is selected. However, the settings of such dimension may be appropriately changed according to design, and the invention is not limited thereto. In addition, according to the settings of such dimension, the intensity of the occurring surface plasmon is varied.

In addition, in the blood-pressure sensor 2, the sensor described in FIG. 1 and peripheral circuits thereof are disposed as illustrated in FIG. 2E. In the blood-pressure sensor 2, the sensor unit 11 and the power-reception photoelectric conversion element 16 are disposed to be aligned at the substantially center of the main plane of the one-chip substrate. In the transverse side thereof, in an aligned shape, the sensor power source unit 15 is disposed near the power-reception photoelectric conversion element 16, and the sensor controller 12 and the data transmitting unit 13 are disposed near the sensor unit 11. In addition, the data-transmission antenna 14 is disposed to around the outer circumference of the main plane of the substrate.

Next, processes of manufacturing the sensor unit 1 are described with reference to FIGS. 3A to 3G.

First, a solar cell is produced as a photoelectric conversion element in the sensor unit 11. As illustrated in FIG. 3A, as a semiconductor material for the substrate 5, a material having the highest efficiency according to a wavelength range of receiving light is selected. The substrate 5 according to the embodiment is configured by using a single crystalline silicon substrate having a thickness of from 100 to 250 μm (p type semiconductor, CZ, plane index 100, and resistivity: 0.1 to 10 Ωcm).

As impurities, phosphorus (P) as dopants is diffused through thermal diffusion so that a desired concentration is obtained. As a result, a pn junction structure is formed.

Next, as illustrated in FIG. 3B, a rear-surface charge collecting layer 7 is formed on the rear surface of the substrate 5 through sputtering by using a material having a good conductivity, for example, aluminum as a target. After that, as illustrated in FIG. 3C, after a mask 31 is formed on the surface of the substrate 5, a charge collecting layer 6 is formed to surround the portion of the grating structures, which is to be the light-receiving unit, by using a selective CVD method or the like. The charge collecting layer 6 may be formed by using a formation method such as sputtering and etching using a photolithography technology.

Next, a process of forming the structures 3 which induces the surface plasmons is described.

FIG. 3D is an enlarged diagram illustrating a portion C in the grating structure illustrated in FIG. 3C. As illustrated in FIG. 3D, after the substrate 5 is UV-cleaned (illuminated with UV light having a wavelength of 172 [nm] and luminance of 10 [mW/cm²] for 10 minutes), gold (Au) sputtering is performed on the substrate, so that an Au layer 33 (3) having a thickness of 150 nm is formed.

A positive type resist for electron lithography (trade name: Zep-520a, manufactured by ZEON CORPORATION) is spin-coated on the Au layer 33 at 4000 rpm, so that a resist layer (thickness: 200 nm) is formed. After that, a desired metal pattern is drawn at a dose rate of, for example, 1.2 μC/cm² by using an electron beam exposure apparatus with an accelerating voltage of 100 kV, and development is performed, so that a resist pattern 32 is performed.

Next, as illustrated in FIG. 3E, the exposed Au layer is etched and removed by dry or wet etching, and patterning according to the resist pattern 32 is performed. By the patterning, the structures 3 are formed. In the embodiment, the structures 3 are disposed in a matrix shape. With respect to the a (=L/2) of the structure 3, a blood vessel deforming strain which is (maximum blood pressure value)−(minimum blood pressure value) is set to 10% by using a rigorous coupled wave analysis (RCWA). As a dimension of the structure, the width b=c is set to 150 [nm]. As a result, as illustrated in later-described FIG. 4, a=0.205 to 0.225 [μm] is obtained. In the embodiment, as an initial pattern before deformation, a=0.205 μm is selected. Such dimension is an exemplary one, and it may be appropriately changed according to difference, specification, and design of the test object.

After that, as illustrated in FIG. 3F, the resulting product is immersed into a resist remover solution, and ultrasonic rinsing is performed, so that resist removing and liftoff are performed. In addition, as illustrated in FIG. 3G, by depositing parylene, a transparent protective layer 8 having a thickness ranging from 0.5 to 2 μm is formed to cover at least all the structures 3. A refractive index of the protective layer 8 constructed with a transparent conductive layer is preferably in a range of from 1.0 to 2.0. In addition, the layer and the like formed on the upper layer including the grating structure, which is to be the light-receiving plane of the photoelectric conversion element, is constructed by using a substantially transparent layer.

Now, the blood-pressure sensor system having the aforementioned configuration is described.

First, a blood-pressure sensor 2 is attached to a main artery blood circulating model to which a blood vessel of a living body is connected. In other words, the blood-pressure sensor is installed on an outer wall of the blood vessel of a test object. With respect to the installation, the blood-pressure sensor 2 is attached closely to the blood vessel from the outer side of the blood vessel, for example, by using a cuff (not shown) or the like having a width (length) suitable for the size (diameter or the like) of the blood vessel. The cuff is configured with a transparent member or the like or with a structure where a portion of the light-receiving plane is exposed so that the light emitted from at least the sensing light-emitting element 21 enters the portion that is to be the light-receiving plane of the solar cell in the blood-pressure sensor 2. In addition, the attachment mechanism is not limited to the cuff, but any belt shape member which can follow the change in the blood pressure value, that is, the expansion or contraction of the blood vessel may be used.

Next, light having a wavelength of from about 700 to about 900 [nm] is illuminated on the blood-pressure sensor 2. At this time, if a change in the blood pressure value occurs, the outer wall of the blood vessel is deformed. Due to the deformation, the value of L (=a/2) in the sensor unit 11 is changed (increased), so that the light absorbance state is changed.

FIG. 4 illustrates a change in the light absorbance (light absorption amount) with respect to the wavelength when, for example, b=c=150 [nm] and the value of a is changed in a range of from 0.205 to 0.225 [μm]. As illustrated above, as the value of a is changed, the light absorbance is most greatly changed near the wavelength of 700 [nm].

Due to the change in the light absorbance, the short circuit current Isc flown at the time of short circuit of the solar cell is monotonously increased.

However, if the short circuit current Isc is monitored, a current is flown. Namely, heat generation occurs. As described above in the problem of the invention, the heat generation is associated with the problem of heat in the conventional deformation gauge. In the embodiment, if the monitoring of the current is not performed, most current is not flown, so that the monitoring of the voltage is used without problem of the heat generation. In other words, the short circuit current Isc is indirectly derived from the following Equation (1) by using the open circuit voltage Voc.

Isc/Io=Exp[Voc·q/kT] (Equation 1)

Namely, the open circuit voltage Voc is continuously measured, so that the ratio α which is a ratio of the short circuit current Isc to the leakage current Io when the solar cell is in a dark state (non-light-receiving time) is continuously calculated. For example, during an arbitrary scheme, a minimum value α0 is detected, and the ratio β (ratio of α to α0) is calculated with reference to the minimum value α0.

As illustrated in FIG. 5, the rate β and the pitch “a” are in a linear relationship, and the deformation amount is estimated from the relationship. The blood pressure value is estimated by comparing the detected deformation amount with information (graphs, equations, or tables) where deformation amounts and actual blood pressure values are set in correspondence with each other in advance. The blood pressure value is expressed by the following equation by using the minimum blood pressure value Pmin. In addition, at the time of measurement, the minimum blood pressure value Pmin is calibrated by using a suitable well-known method (cuff vibration method or the like). In addition, the proportional constant η is a coefficient depending on the characteristics of a material of the blood vessel and is determined at the time of performing the calibration.

Paorta=Pmin+η(1−β) (Equation 2)

With respect to the calibration of pressure, a volume compensation method, a Korotkoff method, or the like is performed. In addition, with respect to the monitoring in the disease such as an arteriosclerosis or at the time of artificial blood vessel replacement, the measurement of pulse pressure may be performed, and the monitoring may be performed by using only the value of β.

Next, the blood-pressure sensor 2 transmits data, that is, the result of calculation to the system main body 20 by using telemetry such as electromagnetic induction. In the embodiment, electromagnetic induction is used for the data transmission. As a communication frequency, by considering a permeability of a living body and a size thereof, a frequency near 433 [MHz] is used. The system main body 20 allows a data receiving unit 22 to receive data through an antenna unit 27 and converts the data into an information signal and outputs the information signal to a data analysis unit 23.

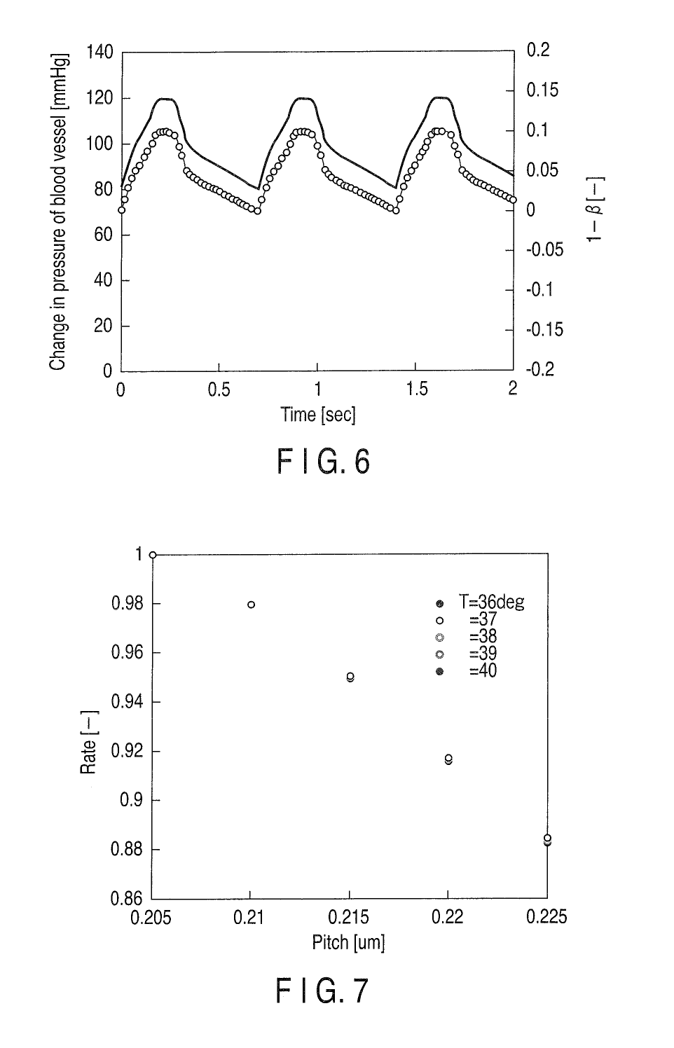

In the data analysis unit 23, a change in the blood pressure value is acquired from the information signal. In other words, as illustrated in FIG. 6, the blood pressure value is calculated by following the change in the pressure of the blood vessel (AOP) [mmHg] (solid thick line) and the amount of strain (1−β) (thin line of circles) from the information signal as time elapses and considering the coefficient η. The calculated blood pressure values may be displayed on the data display unit 24 by using, for example, a graph illustrated in FIG. 6 as well as numerical display. In addition, as illustrated in FIG. 5, at this time, the external temperature T is 37° C.

By performing the aforementioned measurement of the blood pressure by setting the external temperature T to, for example, 36, 37, 38, 39, and 40° C., the relationship between the pitch and the rate illustrated in FIG. 7 can be obtained. As illustrated in FIG. 7, it is found out that, in the case where the blood-pressure sensor according to the embodiment is used at a temperature of from 36 to 40° C. within a physically allowable range, substantially the same characteristics can be obtained, so that the blood-pressure sensor does not depend on most of the external temperature. Although the change in the external temperature T occurs, the sensor is infinitesimally influenced by the temperature, and the same result can be obtained.

As described above, in the blood-pressure sensor in the blood-pressure sensor system according to the embodiment, the surface plasmon resonance is used, the incident form of light is changed according to the deformation, the change in the incident form is converted into electrical data by using the photoelectric conversion element such as a solar cell to obtain the electrical data, so that the information on the deformation of the test object can be acquired. In other words, the open circuit voltage of the solar cell is detected, and the index calculation is performed, so that the displacement of the test object can be measured from the information. Therefore, the output from the blood-pressure sensor is detected based on the open circuit voltage, the result of measurement can be obtained without current flow which causes heat generation. As described above, in a deformation gauge, since a current is flown at the time of performing measurement, heat is generated. Therefore, if a current is not flown, the heat is prevented from being generated. Accordingly, it is possible to solve the problem of heat generation which is the problem in a conventional strain gauge.

In addition, in the blood-pressure sensor system according to the embodiment, in comparison with a conventionally well-known sensor, the dependency on temperature is low, and the measurement can be performed non-invasively and accurately due to the wavelength sensitivity of the surface plasmon resonance (SPR). In addition, it is possible to implement a blood-pressure sensor having a low dependency on temperature. Particularly, since the sensor main body can be manufactured with a very small size and a light weight, it is possible to attach (mount) the sensor main body without stress to a to-be-tested object. In addition, in terms of the power consumption, since a small power is consumed, it is possible to perform the measurement for a long time.

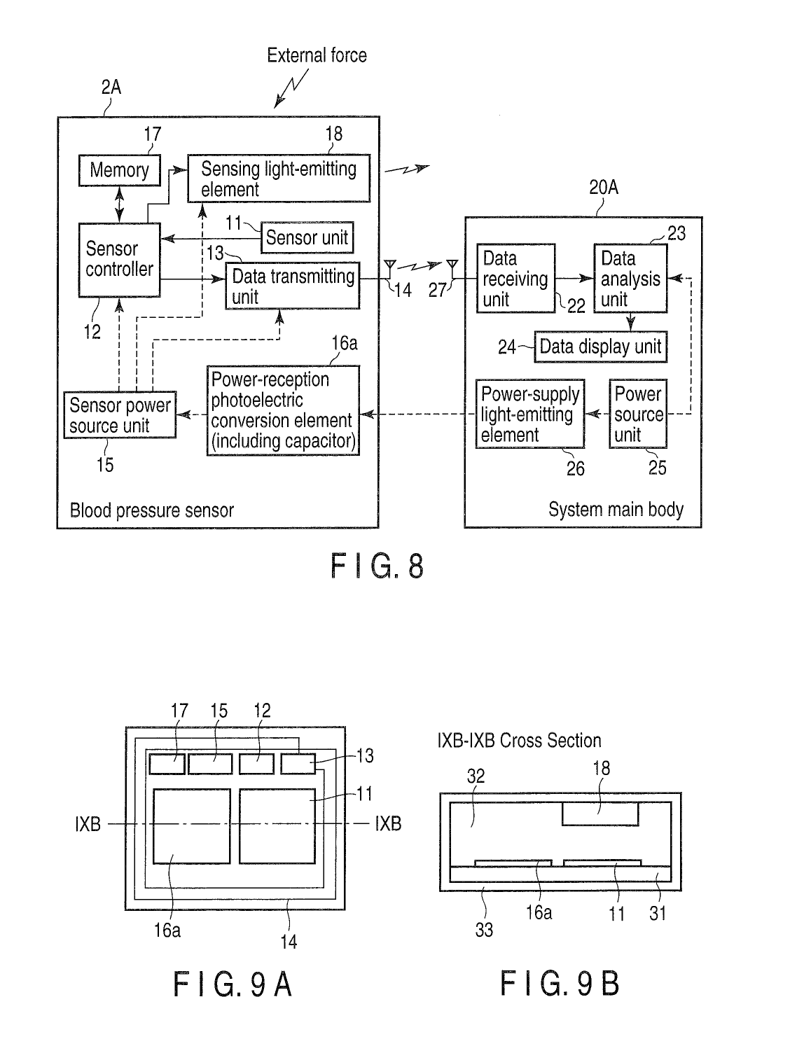

Next, a second embodiment is described. FIG. 8 is a block diagram illustrating a configuration of a blood-pressure sensor system according to the second embodiment. FIG. 9A is a diagram illustrating a configuration of layout of a blood-pressure sensor 2A, and FIG. 9B is a diagram illustrating a configuration of a cross section of the blood-pressure sensor 2A taken along line B-B′ of FIG. 8. In the configuration of the embodiment, the same components as those of the aforementioned first embodiment are denoted by the same reference numerals, and detailed description thereof is omitted.

The embodiment has a configuration where a sensing light-emitting element, which is disposed to the system main body in the first embodiment, is disposed to a blood-pressure sensor. The blood-pressure sensor 2A includes a sensor unit 11, a sensor controller 12, a data transmitting unit 13, a data-transmission antenna 14, a sensor power source unit 15, a power-reception photoelectric conversion element 16a, a memory 17 which sequentially stores measured data in a time sequence and from which the measured data are read in response to the request of the system main body 20A, and a sensing light-emitting element 18 including a light-emitting diode or the like which emits light for measurement to the blood-pressure sensor 2.

In this configuration, the power-reception photoelectric conversion element 16a is provided with a capacitor for storing a power generated through the photoelectric conversion. The stored power is supplied from the sensor power source unit 15 to the sensing light-emitting element 18. The sensor unit 11 has the same configuration as that of the first embodiment, where the structures 3 which induce the surface plasmons are formed.

The sensor controller 12 has a control function of controlling entire the sensor. The sensor controller 12 performs control of temporarily storing the measured data in the memory 17, reading the data from the memory 17 in response to the request of the system main body 20A, and transmitting the data from the data transmitting unit 13 to the system main body.

In addition, the system main body 20A illustrated in FIG. 8 includes an antenna 27 which receives data, a data receiving unit 22 which decodes the received data, a data analysis unit 23 which performs a data analysis and an image signal process to reproduce a blood pressure value, a data display unit 24 including a monitor, and a power source unit 25 which is constructed with a battery or the like.

In the configuration of layout of the blood-pressure sensor 2A illustrated in FIG. 9A, the sensor unit 11 and the power-reception photoelectric conversion element 16a are disposed to be aligned at the substantially center of the main plane of a one-chip substrate 31. In the transverse side thereof, the memory 17, the sensor power source unit 15, the sensor controller 12, and the data transmitting unit 13 are disposed in an aligned shape. In addition, the data-transmission antenna 14 is disposed to curve around the outer circumference of the main plane of the substrate. In addition, the layout is an exemplary one, but the invention is not particularly limited thereto.

In addition, as illustrated in FIG. 9B, the surface (circuit element mounted surface) of the substrate 31 is covered with an elastic deformation material 32, which is made of PDMS (poly dimethyl siloxane) or the like, and a sensing light-emitting element 18 is fixed at a position facing the sensor unit 11 with the elastic deformation material 32 interposed therebetween. These components are surrounded by a coating layer 33 which is made of a material having a high biocompatibility, for example, parylene or the like.

According to the configuration, the sensing light-emitting element 18 emits the light having a wavelength λ, which is an interval set in advance, toward the sensor unit 11. If the sensor unit 11 receives the light, the output value of the charge amount is output through the photoelectric conversion. At this time, if an external force is applied to the sensor 2A, the sensor is deformed, so that the charge amount output from the aforementioned structures 3, which induce the surface plasmons, is changed. As described above, the blood pressure value and the change in the blood pressure value are measured from the change in the charge amount. The obtained measurement data are temporarily stored in the memory 17, and arbitrarily, in response to the request of the system main body 20A, the stored measurement data are read out and transmitted from the data transmitting unit 13 to the system main body 20A.

As described hereinbefore, in the blood-pressure sensor 2A according to the embodiment, since the sensing light-emitting element 18 is disposed within the blood-pressure sensor 2A, although the system main body 20A is not disposed near to the blood-pressure sensor 2A, the sensor can be individually driven so as to measure the blood pressure value.

Next, a third embodiment is described.

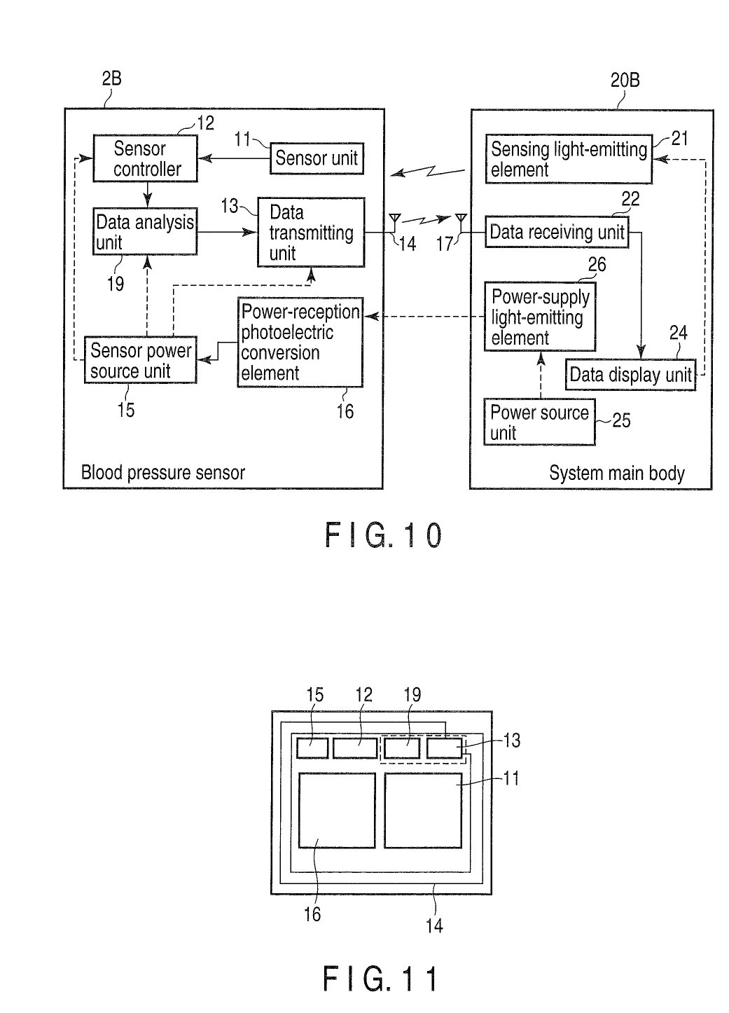

FIG. 10 is a block diagram illustrating a configuration of a blood-pressure sensor system according to the third embodiment. FIG. 11 is a diagram illustrating a configuration of layout of a blood-pressure sensor 2B. In the configuration of the embodiment, the same components as those of the aforementioned first embodiment are denoted by the same reference numerals, and detailed description thereof is omitted.

The embodiment has a configuration where a data analysis unit, which is disposed to the system main body in the first embodiment, is disposed to a blood-pressure sensor.

The blood-pressure sensor 2B includes a sensor unit 11, a sensor controller 12, a data transmitting unit 13, a data-transmission antenna 14, a sensor power source unit 15, a power-reception photoelectric conversion element 16, and a data analysis unit 19.

The sensor controller 12 and the data analysis unit 19 may be configured as an integrated process calculation circuit. The data analysis unit 19 measures the blood pressure value by performing index calculation using the output from the sensing photoelectric conversion element as the open circuit voltage based on the data detected by the sensor unit 11. The data transmitting unit 13 converts the blood pressure value into transmitting data and transmits the transmitting data as an electromagnetic induction wave to the system main body 20B.

In addition, the system main body 20B includes a sensing light-emitting element 21, an antenna 27 which receives data, a data receiving unit 22 which decodes the received data, a data display unit 24 including a monitor, and a power source unit 25 which is constructed with a battery or the like.

According to the third embodiment having the aforementioned configuration, it is possible to obtain the same functions and effects as those of the aforementioned first embodiment.

In the embodiments described hereinbefore, the blood vessel is described as an example of a test object that is to be a measurement object. However, the blood-pressure sensor disclosed in the embodiments can be used as a mechanical sensor. The mechanical sensor is attached closely to a test object of which the shape is changed, namely, of which the shape is expanded, contracted, or curved at the attached position, so that the change in the state can be measured or monitored with the elapse of time. As usage of the mechanical sensor, the mechanical sensor may be easily used as a sensor of sensing a change in a force in another technological field, for example, as a deformation sensor. In addition, the blood-pressure sensor according to the embodiment can also be adapted to a field of a conventional semiconductor strain gauge.

Next, a fourth embodiment is described.

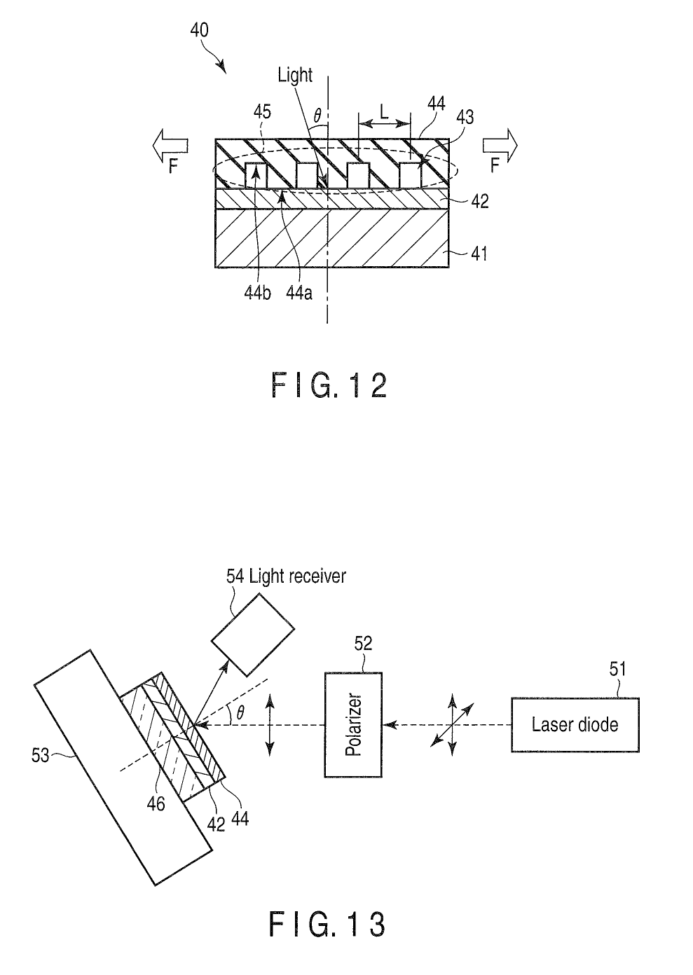

FIG. 12 is a cross-sectional view illustrating a configuration of a blood-pressure sensor system according to the fourth embodiment.

Although the aforementioned blood-pressure sensor according to the first embodiment is constructed with the solar cell (substrate) and the metal grating, in this embodiment, a grating structure having elasticity is employed instead of the metal grating. The blood-pressure sensor according to the embodiment has a configuration where a metal layer (for example, an Au layer) 42 is formed on a photoelectric conversion element, that is, a solar cell 41 which is the same as that in FIGS. 2A and 2C described above, gaps 43 are formed on the metal layer in an equal interval, and an elastic body 44 constructed with, for example, PDMS is disposed. Due to the gaps 43 formed in the elastic body 44, the later-described grating structure is formed. In the description hereinafter, the grating structure formed in the elastic body 44 is referred to as an elastic grating structure 45.

In the blood-pressure sensor 40 illustrated in FIG. 12, the Au layer 42 as a metal layer is formed on the solar cell 41 by using a well-known layer forming technology such as sputtering. Next, the elastic grating structure 45 is formed on the Au layer 42.

In the elastic grating structure 45, a plurality of gaps (spaces) 43 having the same size as a predetermined interval (pitch L) between the elastic grating structure 45 and the Au layer 42 is formed. In other words, the elastic grating structure 45 includes an elastic body plane 44a which is attached to the Au layer 42 and an elastic body plane 44b which is detached from the Au layer 42. In other words, among the light passing through the elastic body 44, the amount of light that reaches the metal layer according to the grating structure 45 is changed.

For example, in the case where the gaps having a pillar shape are disposed in a matrix shape as illustrated in FIG. 2B, the formation of the elastic grating structure 45 in the elastic member 44 may be implemented by using a mold (for example, a metal mold) provided with protrusions which are used to form the gaps 43. Alternatively, the formation may also be performed by cutting out. The elastic grating structure 45 is disposed to be in contact with the Au layer 42 which is formed on the solar cell manufactured by using a well-known semiconductor manufacturing process, and the formed elastic member 44 is separately fixed thereto. As the fixing method, any well-known technology may be used. For example, an adhesive may be used, or dissolving and welding may be used.

Now, characteristics of a solar cell using the surface plasmon resonance effect of the blood-pressure sensor 40 having the aforementioned configuration according to the embodiment are described.

First, the surface plasmon resonance effect in the blood-pressure sensor according to the embodiment is described. In the embodiment, the blood-pressure sensor includes a substrate (solar cell), a metal layer (Au layer), and an elastic body having an elastic grating structure constructed with an arbitrary elastic material (PDMS). In the configuration, a wave number of incident light, which occurs on the surface of the metal layer, is expressed by the following Equation 3.

$${k}_{in} = \frac{ω \sqrt{{ɛ}_{m}}}{c} sin θ + \frac{2 π n}{L} n = \pm 1 , \pm 2 , \pm 3 … \qquad ( Equation 3 )$$

Herein, εₘ is a dielectric constant of surroundings; L is a pitch of the grating structure (refer to FIG. 12); θ is an incident angle; c is the speed of light; and ω is an angular velocity. Equation 1 is satisfied without limitation in a material constituting the grating structure. On the other hand, a wave number of surface plasmon, which occurs on the surface of the metal layer, is expressed by the following Equation 4.

$${k}_{SP} = \frac{ω}{c} \sqrt{\frac{{ɛ}_{m} ɛ ( ω )}{{ɛ}_{m} + ɛ ( ω )}} \qquad ( Equation 4 )$$

Herein, ε(ω) is a dielectric constant of the metal. In this case, the resonance condition is that kᵢₙ−kₛₚ. Therefore, if there is a change in the wave number caused by a change in L on the surface of the metal layer, it can be understood from Equation 4 that there is a change in the surface plasmon resonance effect. Accordingly, the configuration of the embodiment satisfies the requirements.

Hereinafter, occurrence of the surface plasmon resonance in the sensor structure of the blood-pressure sensor according to the embodiment is described.

FIG. 13 illustrates a system for detecting an amount of reflecting light (output of reflecting light) with respect to an angle θ of incident light in the elastic grating structure 45, which is formed on the metal layer in the embodiment, and measuring an angle dependency of a solar cell by using a laser light.

The system includes a laser diode 51 which emits the laser light having a wavelength of, for example, λ=675 nm, a polarizer 52 which performs TM polarization on TM laser light, a sample table 53 on which a test sample described later is mounted so as to be set with a desired angle and to be rotated, and a light receiver 54 which is constructed with a photo-detector or the like and which receives reflecting light from the test sample and outputs an output value according to the intensity of the received reflecting light.

As test samples, a first sample, which is a laminate structure of a stripe-shaped elastic grating structure 45, a glass 46, and a metal layer 42 as illustrated in FIG. 16A, and a second sample, which is a laminate structure of an elastic grating structure 45, a metal layer 42, and a glass 46 as illustrated in FIG. 16B, are used.

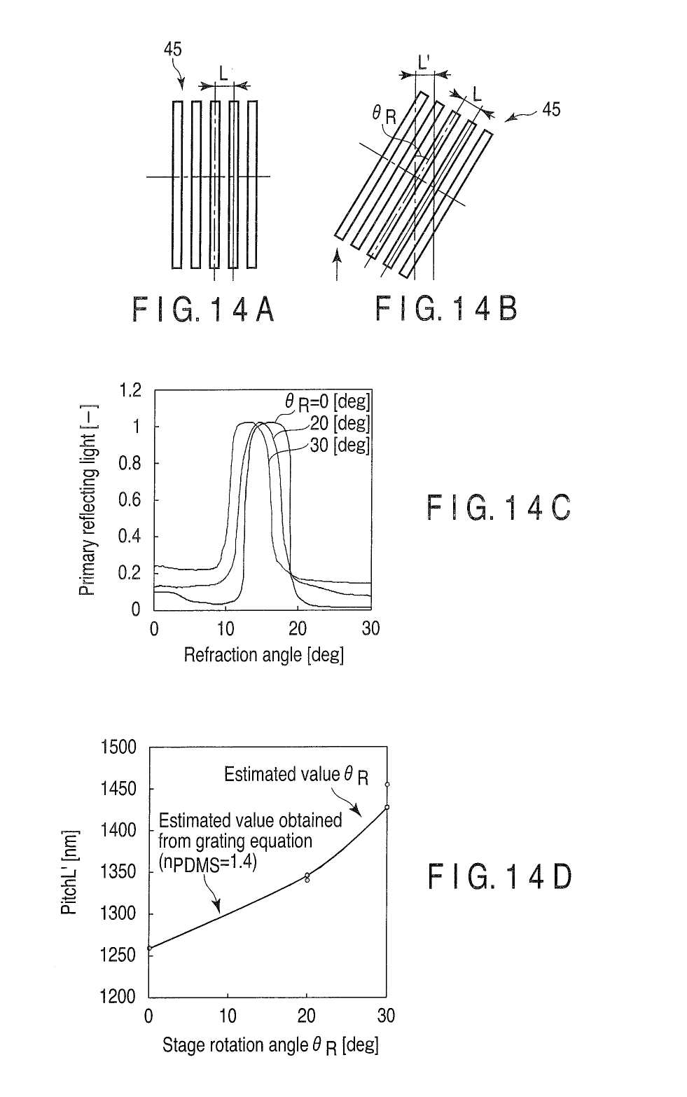

In the system, the first sample is mounted on the sample table 53, and TM-polarized laser light is illuminated thereon. The sample table 53 is rotated at an arbitrary angle of slope (rotation angle θ_{R}) with respect to the incident angle θ (the angle perpendicular to the sample plane is set to 0°). After that, the output (primary reflecting light output) of the reflecting light from the first sample is detected. Similarly, with respect to the second sample, the output (second reflecting light output) of the reflecting light is detected. As illustrated in FIG. 14C, the output occurring in the first sample is obtained from the reflecting light. FIG. 14C illustrates the outputs and corresponding primary reflecting light as the angle θ_{R} of the slope of the sample table 53 is changed to be 0°, 20°, and 30°. The peak in the output characteristic occurs because the incident light is trapped due to the induction of the plasmons on the metal layer 42 caused by the grating structure 45.

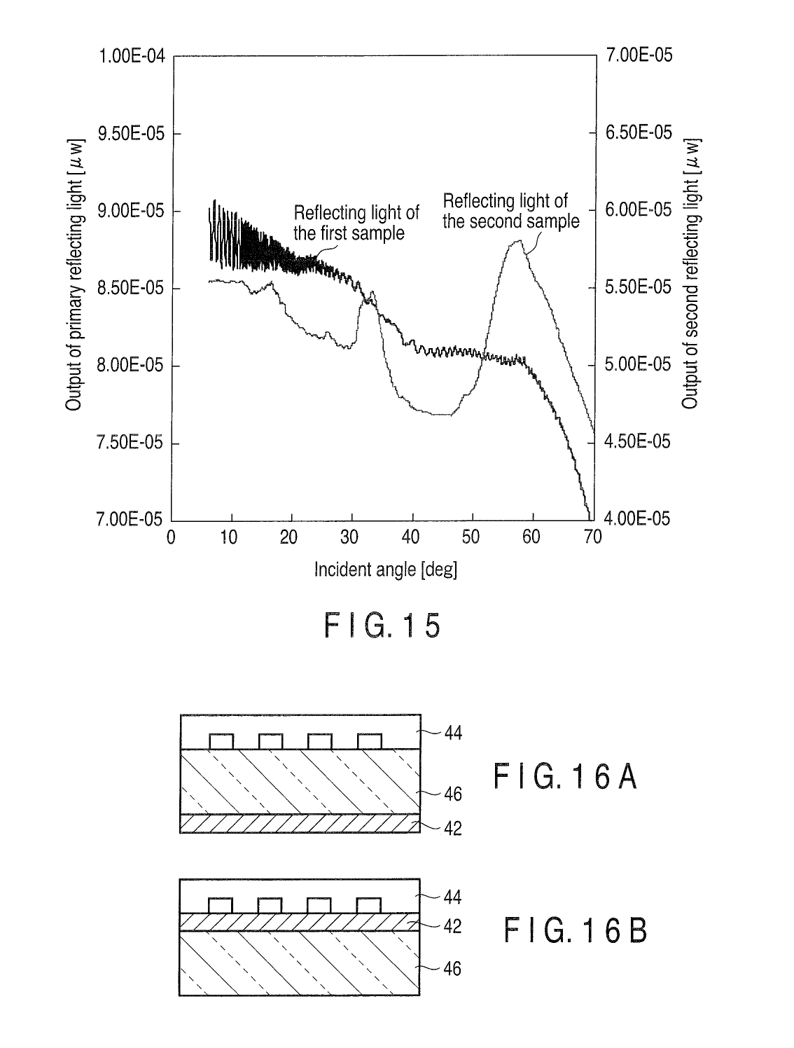

In addition, FIG. 15 is a diagram illustrating outputs of the reflecting lights with respect to incident angles in the first sample and the second sample. As the characteristics of the output of the reflecting light of the first sample (as shown in FIG. 16A), according to the increase in the incident angle θ, the output of the zero-order reflecting light is monotonously decreased. As the result of the detection in the second sample, according to the increase in the incident angle θ, the output of the zero-order reflecting light (reflecting light of second sample (as shown in FIG. 16B)) is large and passes through three peak values (extremum values). As a result, the output of the reflecting light is decreased. From the result, as illustrated in FIG. 15, a peak exists near the incident angle of 55°, so that it may be estimated that the surface plasmon resonance occurs. In addition, the resonance angle is obtained from the distribution of wave number (the above Equations 3 and 4) through simulation by using the average refractive index of air and the elastic body (PDMS) 44 as εₘ−1.2. As a result, it can be understood that resonance exists near a substantially equal angle of 55°.

It can be understood from the results of the above detection and examination that the surface plasmon resonance occurs due to the elastic grating structure 45 according to the embodiment.

Next, operating characteristics of the blood-pressure sensor according to the embodiment are described.

The elastic grating structure 45 according to the embodiment is mechanically expanded and contracted by influence of an applied external force, so that the pitch L is changed. In the case where the pitch L is changed, the diffraction angle is shifted.

A blood-pressure sensor 40 of a second sample (a laminate structure of an elastic grating structure 45, a metal layer (in this case, Al) 42, and a glass 46) illustrated in FIG. 16B is mounted on a table 53 of a test system illustrated in FIG. 13. Next, the table 53 is rotated to an optical axis direction, so that a state that a pitch of the grating structures 45 is optically changed is obtained. In this example, the detection is performed at the rotation angle θ_{R} of 0°, 20°, and 30° illustrated in FIG. 14A. As illustrated in FIG. 14C, it can be understood from the rotation angle θ_{R} that the diffraction angle is shifted.

At this time, with respect to the optical change in the pitch caused by the rotation, an estimated value L1 is obtained from L1=L/cos θ_{R} by using the rotation angle θ_{R} and the pitch L (for example, 1250 [nm]) at the time of production. In addition, an estimated pitch L2 is calculated from the grating equation (λ/L2=εₘ^{1/2}×sin θ_{dif}) by using the angle θ_{dif} of the primary diffracting light reflected from the grating structure. Herein, the refractive index of the elastic member PDMS is set to 1.4.

In this example, at the rotation angle θ_{R} of 0°, the angle θ_{dif} is 27.3°, so that the estimated pitch L2 of 1252 [nm] is obtained. At the rotation angle θ_{R} of 20°, the angle θ_{dif} is 25.2°, so that the estimated pitch L2 of 1339 [nm] is obtained. At the rotation angle θ_{R} of 30°, the angle θ_{dif} is 23.7°, so that the estimated pitch L2 of 1491 [nm] is obtained. By comparing the obtained estimated pitch L1 with the obtained estimated pitches L2, as illustrated in FIG. 14D, it can be understood that the estimated value of the rotation angle θ_{R} and the estimated value of the rotation angle obtained by using the grating equation overlap with each other in the characteristics and are substantially equal to each other.

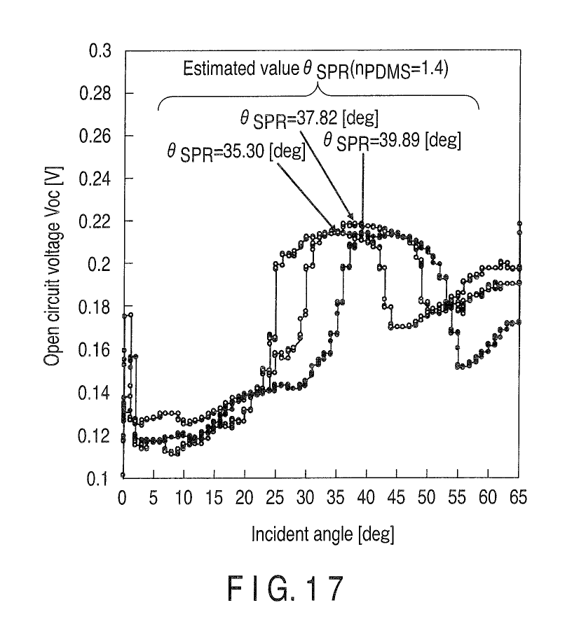

In addition, FIG. 17 illustrates a relationship between the stage rotation angle θ_{R} and the open circuit voltage Voc with respect to the estimated value (angle) θ_{SPR} of occurrence of the surface plasmon resonance. Here, at the rotation angle θ_{R} of 0°, the angle θ_{SPR} is set to 39.89°; at the rotation angle θ_{R} of 20°, the angle θ_{SPR} is set to 37.82°; and at the rotation angle θ_{R} of 30°, the angle θ_{SPR} is set to 35.30°. In this manner, although the shifting occurs according to the rotation angle θ_{R} of the table, the open circuit voltage in the change of the incident angle of light has the same output characteristics.

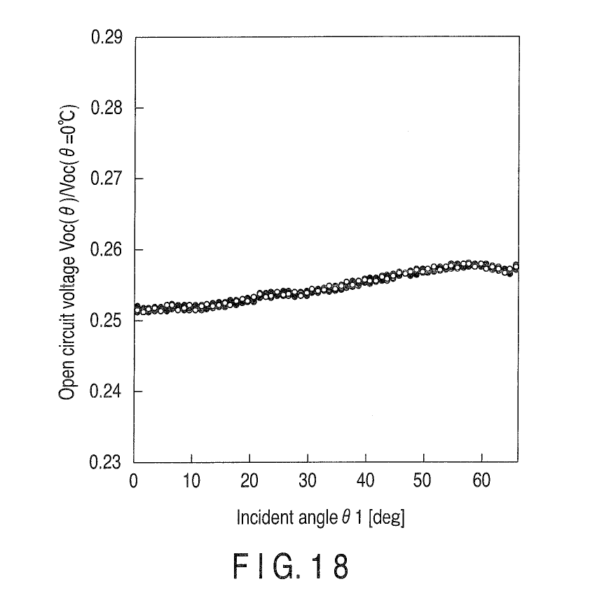

In addition, FIG. 18 illustrates a result of FIG. 17 in the case where a solar cell structure provided with the elastic grating structure 45 is used as a sample. In this case, the value of the open circuit voltage is substantially constant with respect to a change in the incident angle.

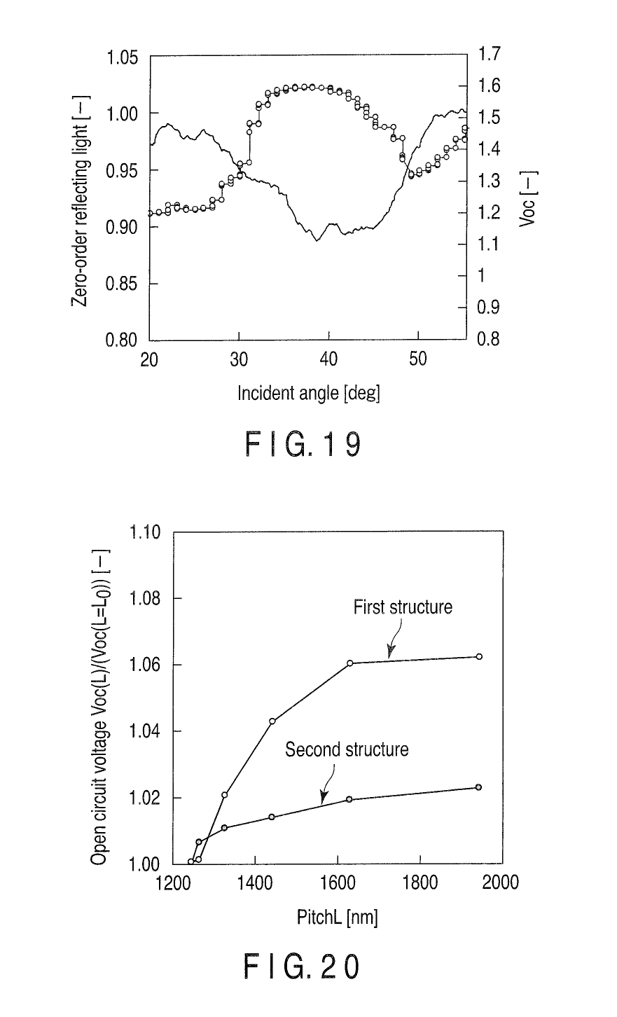

In addition, FIG. 19 illustrates a relationship between the zero-order reflecting light and the open circuit voltage Voc with respect to the incident angle of light in the blood-pressure sensor. In FIG. 19, the open circuit voltage has a convex output characteristic where a peak exists near the incident angle θ of 30°. In other words, it can be understood that the output is changed with respect to the pitch L. The zero-order reflecting light is detected near the angle, so that the decrease in the intensity of light is detected. In other words, it may be considered that, since the surface plasmon resonance state of the metal surface is changed due to the change in the pitch, the change in the amount of light incident to the solar cell is changed according to the change in the surface plasmon resonance state, so that the open circuit voltage is changed.

It can be verified from the above result that, in the case where the change in the pitch occurs according to the mechanical expansion or contraction caused by an external force to the elastic body 44, since the change is equivalent to the optical change in the pitch according to the aforementioned detection, in the elastic grating structure 45 according to the embodiment, the surface plasmon resonance occurs, and the open circuit voltage is changed according to the optical change in the pitch.

Therefore, firstly, since a PDMS elastic body is used for the grating structure, deformation of about 10% or more can be measured. Accordingly, in comparison with the grating structure made of a metal formed on a silicon substrate according to the aforementioned first embodiment, larger deformation can be measured. In other words, the blood-pressure sensor according to the embodiment has a configuration where an applied force or deformation is measured for applications. Since the solar cell disposed on the silicon substrate or the like has no elasticity, the externally applied force is limited to a force of breaking the solar cell or less, and the measurable amount of strain is limited to some extent. In general, although a solar cell can be sufficiently used for a blood-pressure sensor, it has limitations in other types of measurement objects. In the embodiment, since the grating is changed by using the deformation of the elastic body, it is possible to widen the range of the measurement objects. In other words, a material that causes large deformation of a test object may also be adapted to a sensor.

In addition, secondly, since the grating structure is formed in an elastic body, the manufacturing thereof can be performed by using a mold or a mechanical cutting process. Therefore, a general-purpose manufacturing technology can be used, and the manufacturing process thereof can be easily and simply performed. In other words, a semiconductor manufacturing technology (etching process technology) for forming the metal grating structure may not be used for implementing the invention. Thirdly, the embodiment can be easily applied to a light trapping structure of a solar cell.

Next, a modified example of the fourth embodiment is described.

In the aforementioned fourth embodiment, the example where the laser light having no divergence is irradiated as a light source to perform the measurement is described. However, the modified example relates to an example where a light source of emitting diffused light is employed. FIG. 20 illustrates a relationship between an open circuit voltage and a pitch L in the case where the diffused light is used as the light source in a first structure (a laminate structure of an elastic grating structure, a metal layer (in this case, Al), and a solar cell) and a second structure (a laminate structure of an elastic grating structure 45 and a solar cell) similarly to the second sample. In this case, the diffused light source is used as the light source, and an angle of rotation of a stage θ_{R} is set to 0°.

It can be understood from FIG. 20 that the first structure has a larger change in the open circuit voltage according to a change in the pitch. Therefore, similarly to the laser light, the diffused light causes the surface plasmon resonance, and the open circuit voltage changes depending on a change in the optical pitch.

The elastic grating structure according to the fourth embodiment is applied not only to the sensor but also the solar cell. In the embodiment, the wavelength absorbed among wavelengths of the externally illuminated light varies according to the change in the dimension of pitch. Therefore, the dimension of the pitch of the grating is adjusted by performing expansion or contraction in accordance with the wavelength of the light source used for power generation, so that it is possible to increase efficiency of power generation.

According to the embodiments of the invention, it is possible to provide a blood-pressure sensor system capable of measuring a blood pressure value non-invasively and accurately with respect to a blood vessel and having a low dependency on temperature.

The embodiments described hereinbefore include the following features of the invention.

(1) A mechanical sensor system comprising a sensor unit including:

a photoelectric conversion element which converts light incident onto a light-receiving plane into an electrical signal and outputs the electrical signal; and

a plurality of structures which are separately disposed on the light-receiving plane at predetermined intervals and each of which is made of a conductive material that induces surface plasmons;

in which when the sensor unit is attached to a test object, the interval of the structures are changed to expanding an opening or to be tapered (be widened or narrowed) according to expansion or contraction of the test object due to a change in the shape of the test object, so that a form of light incident to the structures is changed to cause a change in the output from the photoelectric conversion element, and

in which an index calculation is performed on the output measured as an open circuit voltage, and a pressure value as a result of the measurement is obtained using the result of the index calculation and information where the indexes and the pressure values are associated with each other.

(2) A mechanical sensor system comprising: a mechanical sensor including a sensor unit including a sensing photoelectric conversion element which converts light-for-measurement incident to a light-receiving plane into an electrical signal and outputs the electrical signal and a plurality of structures which are separately disposed on the light-receiving plane at predetermined intervals and each of which is made of a conductive material that induces surface plasmons; a transmitting unit which transmits data detected by the photoelectric conversion element as an electromagnetic induction wave; and a power-supply photoelectric conversion element which converts light-for-power that is incident to the light-receiving plane into a power and outputs the power as a driving power source, and

a system main body including a first light-emitting element which emits light-for-measurement to the sensor unit; a second light-emitting element which emits the light-for-power to the power-supply photoelectric conversion element; a data receiving unit which receives the data which is constructed with the electromagnetic induction wave transmitted from the transmitting unit; and a data analysis unit which measures an exerted force by performing index calculation by using the output from the sensing photoelectric conversion element as an open circuit voltage based on the received data,

in which when the mechanical sensor is attached to an outer wall of a test object, the interval of the structures are changed to expanding an opening or to be tapered according to expansion or contraction of the test object due to a change in the shape of the test object, so that a form of light incident to the structures is changed to cause a change in the output from the photoelectric conversion element, and

in which the data analysis unit measures the force by measuring the output as an open circuit voltage and performing the index calculation.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A blood-pressure sensor system comprising a blood-pressure sensor including:
a photoelectric conversion element which converts light incident to a light-receiving plane into an electrical signal and outputs the electrical signal; and
a plurality of structures which are separately disposed on the light-receiving plane at predetermined intervals and each of which is made of a conductive material that induces surface plasmons,
wherein when the blood-pressure sensor is attached to an outer wall of a blood vessel, the intervals of the structures are changed to expanding an opening or to be tapered according to expansion or contraction of the blood vessel due to a change in the blood pressure of the blood vessel, so that a form of light incident to the structures is changed to cause a change in the output from the photoelectric conversion element, and
index calculation is performed on the output measured as an open circuit voltage, and a blood pressure value is obtained using a result of the index calculation and information where the indexes and the blood pressure values are associated with each other.

2. The blood-pressure sensor system according to claim 1,
wherein in the index calculation, a short circuit current Isc is indirectly derived from a following Equation 1 by using the open circuit voltage Voc,
Isc/Io=Exp[Voc·q/kT] (Equation 1)
a ratio α of the obtained short circuit current Isc to a leakage current Io which flows when a solar cell is in a dark state with no light received is calculated, a ratio β (a ratio of α:α0) is calculated with reference to a minimum α0 which is previously obtained, an amount of strain is estimated from (1−β), and the blood pressure value is estimated from the following Equation 2, and
Paorta=Pmin+η(1−β) (Equation 2)
in Equation 1 and Equation 2, Voc represents an open circuit voltage which is generated when a load is not connected to a solar cell, Isc represents a short circuit current which flows when the photoelectric conversion element is short-circuited, Io represents a leakage current which flows when the photoelectric conversion element is in a dark state, k represents Boltzmann constant, T represents a temperature, q represents elementary charge, α represents a ratio of Isc to To (Isc/Io), β represents a ratio of α to a minimum α0 of the ratio α, Pmin represents a minimum value of blood pressure, Paorta represents an internal pressure of blood vessel (blood pressure), and η represents a pressure conversion coefficient (coefficient constructed with Young's modulus of blood vessel and Poisson's Ratio).

3. The blood-pressure sensor system according to claim 1, wherein the blood-pressure sensor comprises a protective layer that has a refractive index of 1.0 to 2.0 and is configured to cover at least the structures.

4. A blood-pressure sensor system comprising:
a blood-pressure sensor; and
a system main body,
the blood-pressure sensor including:
a sensor unit including a sensing photoelectric conversion element which converts light-for-measurement incident to a light-receiving plane into an electrical signal and outputs the electrical signal and a plurality of structures which are separately disposed on the light-receiving plane at predetermined intervals and each of which is made of a conductive material that induces surface plasmons;
a transmitting unit which transmits data of a detection result from the photoelectric conversion element as an electromagnetic induction wave; and
a power-supply photoelectric conversion element which converts light-for-power incident to the light-receiving plane into a power and outputs the power as a driving power source, and
the system body including:
a first light-emitting element which emits the light-for-measurement to the sensor unit;
a second light-emitting element which emits the light-for-power to the power-supply photoelectric conversion element;
a data receiving unit which receives the data including the electromagnetic induction wave transmitted from the transmitting unit; and
a data analysis unit which measures a blood pressure value by performing an index calculation by using the output from the sensing photoelectric conversion element as an open circuit voltage based on the received data,
wherein when the blood-pressure sensor is attached to an outer wall of a blood vessel, the intervals of the structures are changed to expanding an opening or to be tapered according to expansion or contraction of the blood vessel due to a change in the blood pressure of the blood vessel, so that a form of light incident to the structures is changed to cause a change in the output from the photoelectric conversion element, and
the data analysis unit measures the blood pressure value by measuring the output as an open circuit voltage and performing the index calculation.

5. The blood-pressure sensor system according to claim 4,
wherein in the index calculation, a short circuit current Isc is indirectly derived from a following Equation 1 by using the open circuit voltage Voc,
Isc/Io=Exp[Voc·q/kT] (Equation 1)
a ratio α of the obtained short circuit current Isc to a leakage current Io which flows when the solar cell is in a dark state with no light received, a ratio β(α:α0) is calculated with reference to a minimum α0 which is previously obtained, an amount of strain is estimated from (1−β), and the blood pressure value is estimated from a following Equation 2, and
Paorta=Pmin+η(1−β) (Equation 2)
in Equation 1 and Equation 2, Voc represents an open circuit voltage which is generated when a load is not connected to a solar cell, Isc represents a short circuit current which flows when the photoelectric conversion element is short-circuited, Io represents a leakage current which flows when the photoelectric conversion element is in a dark state, k represents Boltzmann constant, T represents a temperature, q represents elementary charge, α represents a ratio of Isc to Io (Isc/Io), β represents a ratio of α to a minimum α0 of the ratio α, Pmin represents a minimum value of blood pressure, Paorta represents an internal pressure of blood vessel (blood pressure), and η represents a pressure conversion coefficient (coefficient constructed with Young's modulus of blood vessel and Poisson's Ratio).

6. The blood-pressure sensor system according to claim 4, wherein the blood-pressure sensor comprises a protective layer that has a refractive index of 1.0 to 2.0 and is configured to cover at least the structures.

7. A blood-pressure sensor system comprising:
a blood-pressure sensor; and
a system body,
the blood-pressure sensor including:
a sensor unit that includes a sensing photoelectric conversion element which converts light-for-measurement incident to a light-receiving plane into an electrical signal and outputs the electrical signal and a plurality of structures which are separately disposed on the light-receiving plane at predetermined intervals and each of which is made of a conductive material that induces surface plasmons;
a first light-emitting element which emits the light-for-measurement to the sensor unit;
a memory which sequentially stores data of a detection result by the photoelectric conversion element in a time sequence;
a transmitting unit which transmits the data read from the memory as an electromagnetic induction wave; and
a power-supply photoelectric conversion element which converts light-for-power incident to the light-receiving plane into a power and outputs the power as a driving power source, and
the system main body including:
a second light-emitting element which emits the light-for-power to the power-supply photoelectric conversion element;
a data receiving unit which receives the data including the electromagnetic induction wave transmitted from the transmitting unit; and
a data analysis unit which measures a blood pressure value by performing an index calculation by using the output from the sensing photoelectric conversion element as an open circuit voltage based on the received data,
wherein when the blood-pressure sensor is attached to an outer wall of a blood vessel, the intervals of the structures are changed to expanding an opening or to be tapered according to expansion or contraction of the blood vessel due to a change in the blood pressure of the blood vessel, so that a form of light incident to the structures is changed to cause a change in the output from the photoelectric conversion element, and
the data analysis unit measures the blood pressure value by measuring the output as an open circuit voltage and performing the index calculation.

8. A blood-pressure sensor system comprising:
a blood-pressure sensor; and
a system body,
the blood-pressure sensor including:
a sensor unit that includes a sensing photoelectric conversion element which converts light-for-measurement incident to a light-receiving plane into an electrical signal and outputs the electrical signal and a plurality of structures which are separately disposed on the light-receiving plane at predetermined intervals and each of which is made of a conductive material that induces surface plasmons;
a data analysis unit which measure a blood pressure value by performing an index calculation by using an output from the sensing photoelectric conversion element as an open circuit voltage based on the data of a detection result by the sensor unit;
a transmitting unit which converts the blood pressure value into transmission data and transmits the transmission data as an electromagnetic induction wave; and
a power-supply photoelectric conversion element which converts light-for-power incident to the light-receiving plane into a power and outputs the power as a driving power source, and
the system main body including:
a first light-emitting element which emits the light-for-measurement to the sensor unit;
a second light-emitting element which emits the light-for-power to the power-supply photoelectric conversion element;
a data receiving unit which receives the data including the electromagnetic induction wave transmitted from the transmitting unit; and
wherein when the blood-pressure sensor is attached to an outer wall of a blood vessel, the intervals of the structures are changed to expanding an opening or to be tapered according to expansion or contraction of the blood vessel due to a change in the blood pressure of the blood vessel, so that a form of light incident to the structures is changed to cause a change in the output from the photoelectric conversion element, and
the data analysis unit measures the blood pressure value by measuring the output as an open circuit voltage and performing the index calculation.

9. A blood-pressure sensor system comprising:
a blood-pressure sensor including a photoelectric conversion element which converts light incident to a light-receiving plane into an electrical signal and outputs the electrical signal, a metal layer which is formed on the light-receiving plane, and elastic structures having a grating structure on the metal layer,
wherein when the blood-pressure sensor is attached to an outer wall of a blood vessel, and the intervals of the structures are changed to expanding an opening or to be tapered according to expansion or contraction of the blood vessel due to a change in the blood pressure of the blood vessel, so that a form of light incident to the structures is changed to cause a change in the output from the photoelectric conversion element, and
index calculation is performed on the output measured as an open circuit voltage, and a blood pressure value is obtained based on a result of the index calculation and information where the indexes and the blood pressure values are associated with each other.

10. The blood-pressure sensor system according to claim 9, wherein the grating structure takes a form in which a plurality of gaps are formed at an arbitrary pitch on the surface of the elastic body which is in contact with the metal layer, so that a dimension of the pitch is changed by an externally applied expansion or contraction force.

11. The blood-pressure sensor system according to claim 10, wherein the grating structure is configured in a manner such that the wavelength absorbed among wavelengths of the light that is irradiated from an external source varies depending on a change in the dimension of the pitch.
